(19) United States
(12) Patent Application Publication
(10) Pub. No.: US 2022/0226627 A1
(43) Pub. Date: Jul. 21, 2022

(54) SURGICAL STAPLING DEVICE WITH THERAPEUTIC SUPPOSITORY

(71) Applicant: Covidien LP, Mansfield, MA (US)

(72) Inventor: Olesea Diaz-Chiosa, Naugatuck, CT (US)

(21) Appl. No.: 17/556,173

(22) Filed: Dec. 20, 2021

Related U.S. Application Data
(60) Provisional application No. 63/137,836, filed on Jan. 15, 2021.

(51) Int. Cl. A61M 37/00; A61B 17/115

(52) U.S. Cl. CPC A61M 37/0069; A61B 2017/00893; A61B 17/1155

(57) ABSTRACT

A circular stapling device includes one or more therapeutic-containing suppositories that may be secured to an anastomotic site during an anastomotic procedure to reduce the level of bacterial collagenase and minimize the likelihood of anastomotic leakage.

### CROSS-REFERENCE TO RELATED APPLICATION

This application claims the benefit of and priority to U.S. Provisional Patent Application No. 63/137,836, filed Jan. 15, 2021, the entire contents of which is incorporated by reference herein.

### FIELD

This disclosure generally relates to a surgical stapling device and, more particularly, to a circular stapling device that includes a suppository to inhibit pathogens at an anastomotic site.

### BACKGROUND

Circular stapling devices for performing surgical procedures such as anastomoses are well known. In an anastomosis procedure, two ends of organ sections are joined with the circular stapling device. Typically, circular stapling devices include a handle assembly, an elongated shaft or adapter assembly, a shell assembly including a staple cartridge, and an anvil assembly that is mountable to the adapter assembly in movable relation to the shell assembly. In use, opposed tissue end margins of the organ sections are clamped between an anvil head of the anvil assembly and the staple cartridge and the device is fired to drive an annular array of staples from the staple cartridge through the tissue end margins of the organ sections for deformation against the anvil head. An annular knife positioned within the shell assembly is advanced to core or remove organ tissue interior of the staples to clear an internal tubular passage of the organ sections.

Complications during anastomoses procedures may result in a need for further operation, permanent ostomy, and even death. One complication is anastomotic leakage. The risk of anastomotic leakage is multi-factorial and may be affected by patient comorbidities, chemotherapy, the presence of microbiome pathogens, and stapling technique. The presence of microbiomes increases the likelihood of anastomotic leakage. More particularly, microbiome pathogens including Serratia marcescens and Pseudomonas aeruginosa produce collagenase, an enzyme that breaks down peptide bonds of collagen which may prevent wound remodeling at the anastomotic site and result in anastomotic leakage.

A continuing need exists for a circular stapling device that can minimize the existence of microbiome pathogens at an anastomotic site.

### SUMMARY

This disclosure is directed to a circular stapling device for performing anastomoses. The stapling device includes an end effector that supports one or more suppositories.

One aspect of the disclosure is directed to an end effector including an anvil assembly, a shell assembly, a first suppository, and a second suppository. The anvil assembly includes an anvil shaft and an anvil head. The anvil shaft has a proximal portion and a distal portion. The anvil head is supported on the distal portion of the anvil shaft and includes an annular staple forming surface. The shell assembly includes a shell housing and a staple cartridge. The staple cartridge is supported on the shell housing and includes an annular body and staples. The annular body defines staple slots and includes a tissue engaging surface. The staples are received within the staple slots. The first suppository is supported on the staple forming surface of the anvil head and includes an annular body having a therapeutic agent. The second suppository is supported on the tissue engaging surface of the annular body of the staple cartridge and includes the therapeutic agent.

Another aspect of the disclosure is directed to a circular stapling device including a handle assembly, an adapter assembly, and an end effector. The adapter assembly has a proximal portion coupled to the handle assembly and a distal portion including an anvil retainer. The end effector is supported on the distal portion of the adapter assembly and includes an anvil assembly, a shell assembly, a first suppository, and a second suppository. The anvil assembly includes an anvil shaft and an anvil head. The anvil shaft has a proximal portion coupled to the anvil retainer and a distal portion. The anvil head includes an annular staple forming surface. The shell assembly is supported on the distal portion of the adapter assembly and includes a shell housing and a staple cartridge. The staple cartridge is supported on the shell housing and includes an annular body and staples. The annular body defines staple slots and includes a tissue engaging surface. The staples are received within the staple slots. The first suppository is supported on the staple forming surface of the anvil head and has an annular body including a therapeutic agent. The second suppository is supported on the tissue engaging surface of the annular body of the staple cartridge and includes the therapeutic agent.

Another aspect of the disclosure is directed to a suppository that includes a waxy base and a therapeutic agent. The waxy base defines an annular body and is formed of a material that is solid at room temperature and melts at body temperature. The therapeutic agent is included in the waxy base.

In aspects of the disclosure, the first and second suppositories each include a waxy base having the therapeutic agent.

In some aspects of the disclosure, the first suppository is press-fit onto the staple forming surface of the anvil head of the anvil assembly, and the second suppository is press-fit onto the tissue engaging surface of the staple cartridge.

In certain aspects of the disclosure, the therapeutic agent is an antibiotic.

In aspects of the disclosure, the therapeutic agent is a polyphosphate.

In some aspects of the disclosure, the staple forming surface of the anvil head includes staple forming pockets.

In certain aspects of the disclosure, the first and second suppositories are formed of a material that is solid at room temperature and melts at body temperature.

In aspects of the disclosure, the waxy base of the first and second suppositories is formed from glycerin.

Other features of the disclosure will be appreciated from the following description.

### BRIEF DESCRIPTION OF THE DRAWINGS

Various aspects of a circular stapling device are described herein below with reference to the drawings, wherein:

FIG. 1 is a perspective view of a circular stapling device including a manually powered handle assembly according to aspects of the disclosure with the stapling device in an open position;

FIG. 2 is a perspective view of a circular stapling device including an electrically powered handle assembly according to aspects of the disclosure with the stapling device in an open position;

FIG. 3 is an enlarged view of the indicated area of detail shown in FIG. 1;

FIG. 4 is an enlarged view of the indicated area of detail shown in FIG. 2;

FIG. 5 is a side perspective view of an anvil assembly of the circular stapling devices shown in FIGS. 1 and 2 with a suppository separated from the anvil assembly;

FIG. 6 is a perspective view of the distal portion of the circular stapling devices shown in FIGS. 1 and 2 with an anvil assembly of the circular stapling devices removed and a suppository separated from a staple cartridge of the circular stapling device;

FIG. 7 is a view of a portion of a digestive system of a patient after a diseased portion of the colon of the digestive system is resected and two end portions of the resected colon are spaced from each other;

FIG. 8 is a side perspective view of the circular stapling device shown in FIG. 1 positioned within the colon of a patient with the anvil assembly received within one end portion of the colon and a distal portion of the circular stapling device positioned in the other end portion of the colon with the circular stapling device in an open position;

FIG. 9 is a cross-sectional view taken along section line 9-9 of FIG. 8;

FIG. 10 is a side perspective view of the circular stapling device shown in FIG. 1 positioned within the colon of a patient with the anvil assembly received within one end portion of the colon and the distal portion of the circular stapling device positioned in the other end portion of the colon with the circular stapling device in a clamped position;

FIG. 11 is a side cross-sectional view taken through the anastomosed end portions of the colon shown in FIG. 9 with the suppositories coupled to the end portion at the site of the anastomosis; and

FIG. 12 is a side cross-sectional view taken through the anastomosed end portions of the colon shown in FIG. 9 at the site of the anastomosis after the suppositories have melted.

### DETAILED DESCRIPTION

Aspects of the disclosure are now described in detail with reference to the drawings in which like reference numerals designate identical or corresponding elements in each of the several views. As used herein, the term “clinician” refers to a doctor, a nurse, or any other care provider and may include support personnel. Throughout this description, the term “proximal” refers to that portion of the device or component thereof that is closest to the clinician during use of the device in its customary manner and the term “distal” refers to that portion of the device or component thereof that is farthest from the clinician.

This disclosure is directed to a circular stapling device that includes one or more suppositories that include a therapeutic agent. The suppositories are secured to an anastomotic site during an anastomotic procedure to minimize the level of bacterial collagenase at the anastomotic site and minimize the likelihood of anastomotic leakage.

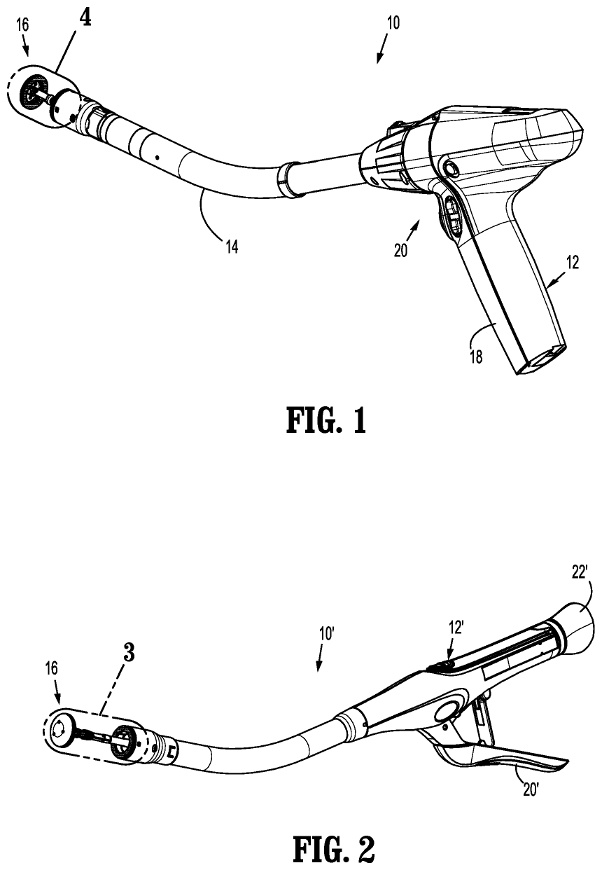

FIG. 1 illustrates a circular stapling device 10 shown generally as stapling device 10 that includes a handle assembly 12, an elongate body or adapter assembly 14 that extends from the handle assembly 12, and an end effector 16 that is coupled to the adapter assembly 14. The handle assembly 12 may be electrically powered and include a motor and associated gears and linkages to control operation of the stapling device 10. The handle assembly 12 includes a stationary grip portion 18 and a plurality of actuation buttons 20 which may be activated to control various functions of the stapling device 10 including, e.g., approximation of the end effector 16 and firing of staples. The stationary grip 18 may support a battery pack (not shown) which powers the handle assembly 12. U.S. Pat. No. 10,327,779 discloses an exemplary powered circular stapling device.

It is also envisioned that the stapling device 10′ (FIG. 2) can include a manually powered handle assembly 12′ having a stationary grip portion 18′, a firing trigger 20′, and an approximation knob 22′. U.S. Pat. No. 10,022,126 (“the '126 patent”) discloses an exemplary manually actuated circular stapling device.

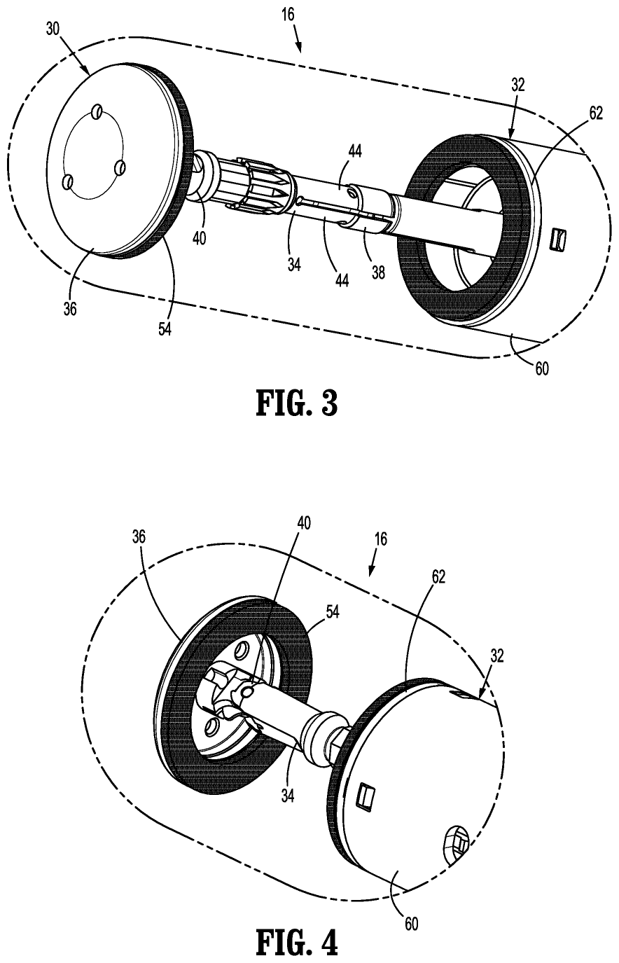

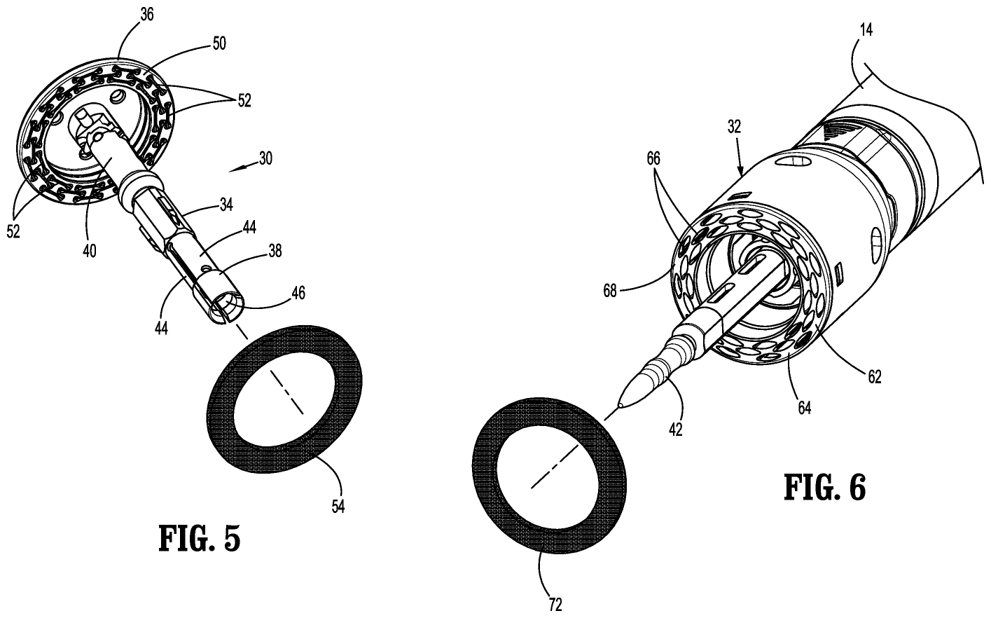

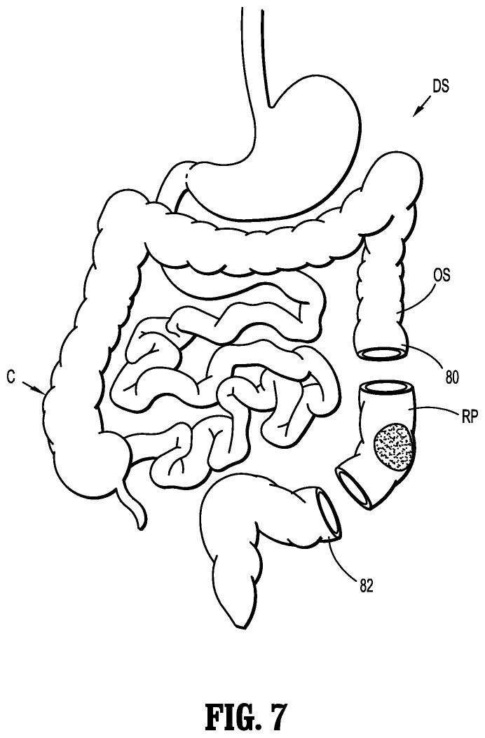

FIGS. 3-6 illustrate the end effector 16 of the stapling devices 10 and 10′ which includes an anvil assembly 30 and a shell assembly 32. The anvil assembly 30 includes an anvil shaft 34 and an anvil head 36. The anvil shaft 34 includes a proximal portion 38 and a distal portion 40. The distal portion 40 supports the anvil head 36. In aspects of the disclosure, the anvil head 30 is pivotably coupled to the anvil shaft 34 and is movable from an operative position (FIG. 5) to a pivoted or tilted position (not shown). In the tilted position, the profile of the anvil head 36 is minimized to facilitate insertion and/or removal of the anvil assembly 30 to and from an organ section “OS”. (FIG. 7). The proximal portion 38 of the anvil shaft 34 is adapted to releasably engage an anvil retainer 42 (FIG. 6) of the stapling device 10 (FIG. 1). In aspects of the disclosure, the proximal portion 38 of the anvil shaft 34 includes resilient fingers 44 that define a longitudinal bore 46 (FIG. 5) that receive the anvil retainer 42 (FIG. 6) of the stapling device 10 to couple the anvil assembly 30 to the anvil retainer 42. For a detailed description of an anvil shaft and anvil retainer suitable for use with the stapling device 10, see the '126 patent.

The anvil head 36 of the anvil assembly 30 includes an annular staple forming surface 50 (FIG. 5) that defines a plurality of staple forming pockets 52. In aspects of the disclosure, the staple forming pockets 52 are formed in annular rows about the staple forming surface 50. The staple forming surface 50 supports an annular suppository 54. In aspects of the disclosure, the annular suppository 54 is formed of a material having a waxy base, e.g., glycerin or similar material, that includes a therapeutic agent, e.g., an antibiotic or polyphosphate. In aspects of the disclosure, the waxy base is formed of a material that is solid at room temperature and melts at body temperature such that when secured within an organ section “OS” (FIG. 7) of a patient, the waxy base melts to deliver the therapeutic agent to the tissue within the organ section “OS”. In aspects of the disclosure, the annular suppository 54 is press-fit onto the staple forming surface 50 of the anvil head 36 of the anvil assembly 30. Alternately, it is envisioned that the annular suppository 54 can be secured to the staple forming surface 50 of the anvil head 36 of the anvil assembly 30 using other known techniques or devices including adhesives.

The shell assembly 32 includes a shell housing 60 that supports a staple cartridge 62. The staple cartridge 62 is supported within a distal portion of the shell housing 60 and includes an annular body 64 that defines staple slots 66 (FIG. 6) and includes a tissue contact surface 68 (FIG. 6). Each of the staple slots 66 receives a staple 70 (FIG. 11). In aspects of the disclosure, the staple slots 66 are arranged in annular rows that are positioned about the annular body 64 of the staple cartridge 62. When the stapling device 10 is approximated by retracting the anvil retainer 42 (FIG. 6) into the shell assembly 32, the staple forming surface 50 of the anvil head 36 is moved into juxtaposed alignment with the tissue contact surface 68 of the staple cartridge 62 to a clamped position. In the clamped position, the staple slots 66 of the staple cartridge 62 are aligned with the staple forming pockets 52 of the anvil head 36.

The staple cartridge 62 of the shell assembly 32 supports an annular suppository 72. In aspects of the disclosure, the annular suppository 72 is like the annular suppository 54 and is formed of a material having a waxy base, e.g., glycerin or similar material, that includes a therapeutic agent, e.g., an antibiotic or polyphosphate. In aspects of the disclosure, the waxy base is formed of a material that is solid at room temperature and melts at body temperature such that when secured within an organ section “OS” (FIG. 7) of a patient, the waxy base melts to deliver the therapeutic agent to the tissue within the organ section “OS”. In aspects of the disclosure, the annular suppository 72 is press-fit onto the tissue contact surface 68 of the staple cartridge 50 of the shell assembly 32. Alternately, it is envisioned that the annular suppository 72 can be secured to the tissue contact surface 68 of the staple cartridge 50 of the shell assembly 32 using other known techniques or devices including adhesives.

FIG. 7 illustrates a portion of a digestive system “DS” of a patient in which a portion “RP” of a colon “C” of the digestive system “DS” has been resected such that end portions 80 and 82 of the colon “C” to be anastomosed are positioned in spaced relation to each other.

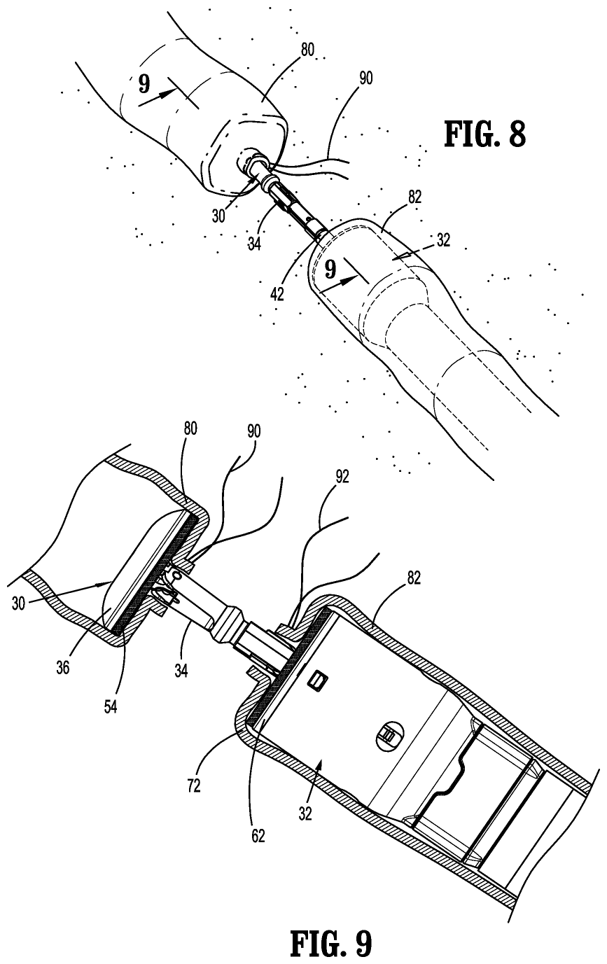

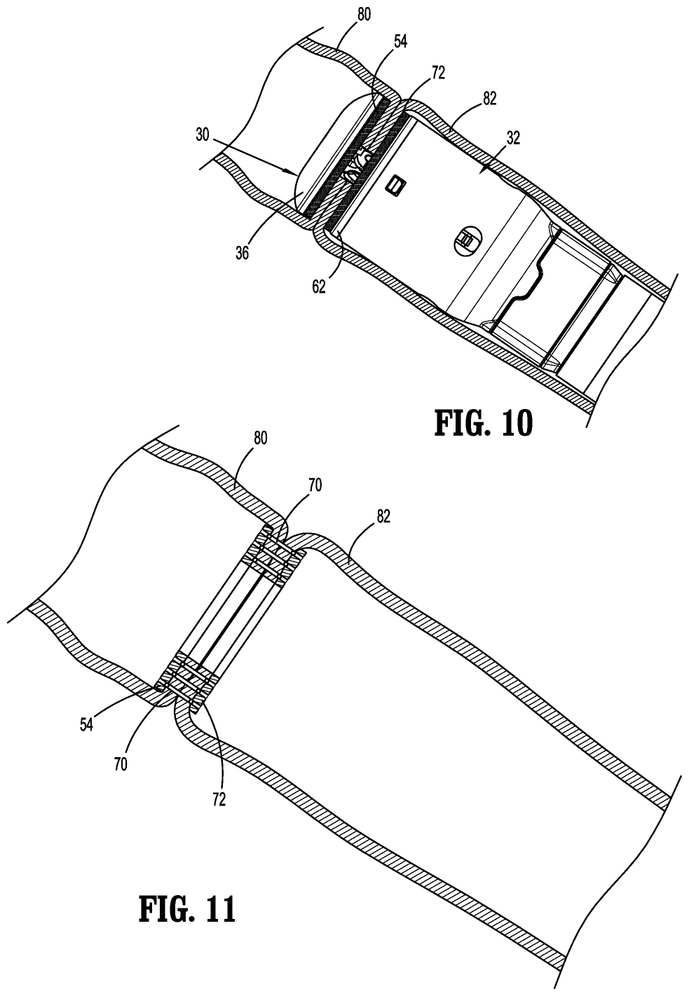

FIGS. 8-11 illustrate an anastomosis procedure using the stapling device 10. During the anastomosis procedure, the anvil head 36 of the anvil assembly 30 is positioned within the end portion 80 of the colon “C” and a purse-string suture 90 is applied to the end portion 80 to secure the end portion 80 about the anvil shaft 34. Next, the shell assembly 32 of the stapling device 10 is positioned within the end portion 82 of the colon “C” and the anvil assembly 30 is coupled to the anvil retainer 42 of the stapling device 10. The end portion 82 of the colon “C” is secured about the anvil retainer 42 using a second purse string suture 92 (FIG. 9). Once the end portions 80 and 82 of the colon “C” are secured to the anvil shaft 34 and the anvil retainer 42, respectively, the stapling device 10 is approximated by retracting the anvil retainer 42 into the shell assembly 32 to move the anvil head 36 of the anvil assembly 30 into juxtaposed opposition with the staple cartridge 62 of the shell assembly 32 to clamp the end portions 80 and 82 between the anvil head 36 and the staple cartridge 62 (FIG. 10).

Once the end portions 80 and 82 of the colon “C” are clamped between the anvil head 36 and the staple cartridge 62, the stapling device 10 can be fired to eject the staples 70 from the staple cartridge 62 through the suppositories 54 and 72 and through the end portions 80 and 82 of the colon “C” to join the end portions 80 and 82 together (FIG. 11) and secure the suppositories 54 and 72 to the end portions 80 and 82 of the colon “C”. As shown in FIG. 11, when the staples 70 are fired into the suppositories, the staples 70 move through the suppositories 54 and 72 to hold the end portions 80 and 82 tightly together.

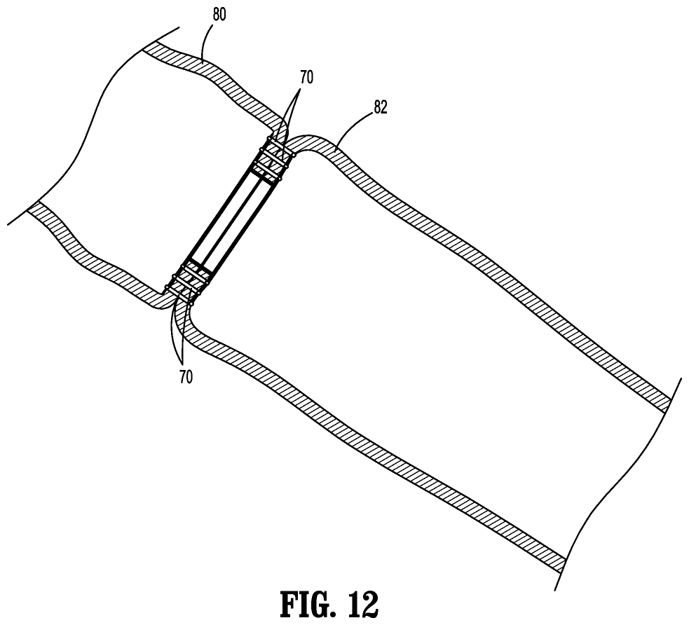

When the suppositories 54 and 72 enter the patient's body, the suppositories 54 and 72 begin to melt and the therapeutic agent within the suppositories 54 and 72 is eluted and absorbed into the mucosa within the anastomosed colon “C” (FIG. 11). Eventually, the suppositories 54 and 72 will melt completely such that only the staples 70 remain at the site of the anastomosis (FIG. 12). The therapeutic agent, e.g., antibiotic, delivered to the location of the anastomotic site will combat pathogen bacteria to minimize the level of bacterial collagenase and minimize the likelihood of anastomotic leakage.

Although the circular stapling device 10 is described to include a suppository on both the anvil assembly and the shell assembly, it is envisioned that only one suppository may be provided on one or the other of the anvil and shell assemblies.

Persons skilled in the art will understand that the instruments and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. An end effector comprising:
an anvil assembly including an anvil shaft and an anvil head, the anvil shaft having a proximal portion and a distal portion, the anvil head supported on the distal portion of the anvil shaft and including an annular staple forming surface;
a shell assembly including a shell housing and a staple cartridge, the staple cartridge supported on the shell housing and including an annular body and staples, the annular body defining staple slots and including a tissue engaging surface, the staples received within the staple slots;
a first suppository supported on the staple forming surface of the anvil head, the first suppository having annular body including a therapeutic agent; and
a second suppository supported on the tissue engaging surface of the annular body of the staple cartridge, the second suppository including the therapeutic agent.

2. The end effector of claim 1, wherein the first and second suppositories each include a waxy base having the therapeutic agent.

3. The end effector of claim 2, wherein the first suppository is press-fit onto the staple forming surface of the anvil head of the anvil assembly, and the second suppository is press-fit onto the tissue engaging surface of the staple cartridge.

4. The end effector of claim 2, wherein the therapeutic agent is an antibiotic.

5. The end effector of claim 2, wherein the therapeutic agent is a polyphosphate.

6. The end effector of claim 2, wherein the staple forming surface of the anvil head includes staple forming pockets.

7. The end effector of claim 2, wherein the first and second suppositories are formed of a material that is solid at room temperature and melts at body temperature.

8. The end effector of claim 7, wherein the waxy base of the first and second suppositories is formed from glycerin.

9. A circular stapling device comprising:
a handle assembly;
an adapter assembly having a proximal portion coupled to the handle assembly and a distal portion, the distal portion including an anvil retainer; and
an end effector supported on the distal portion of the adapter assembly, the end effector including:
an anvil assembly including an anvil shaft and an anvil head, the anvil shaft having a proximal portion coupled to the anvil retainer and a distal portion, the anvil head supported on the distal portion of the anvil shaft and including an annular staple forming surface;
a shell assembly supported on the distal portion of the adapter assembly, the shell assembly including a shell housing and a staple cartridge, the staple cartridge supported on the shell housing and including an annular body and staples, the annular body defining staple slots and including a tissue engaging surface, the staples received within the staple slots;
a first suppository supported on the staple forming surface of the anvil head, the first suppository having annular body including a therapeutic agent; and
a second suppository supported on the tissue engaging surface of the annular body of the staple cartridge, the second suppository including the therapeutic agent.

10. The circular stapling device of claim 9, wherein the first and second suppositories each include a waxy base having the therapeutic agent.

11. The circular stapling device of claim 10, wherein the first suppository is press-fit onto the staple forming surface of the anvil head of the anvil assembly, and the second suppository is press-fit onto the tissue engaging surface of the staple cartridge.

12. The circular stapling device of claim 10, wherein the therapeutic agent is an antibiotic.

13. The circular stapling device of claim 10, wherein the therapeutic agent is a polyphosphate.

14. The circular stapling device of claim 10, wherein the staple forming surface of the anvil head includes staple forming pockets.

15. The circular stapling device of claim 10, wherein the first and second suppositories are formed of a material that is solid at room temperature and melts at body temperature.

16. The circular stapling device of claim 15, wherein the waxy base of the first and second suppositories is formed from glycerin.

17. A suppository comprising:
a waxy base defining an annular body, the waxy base formed of a material that is solid at room temperature and melts at body temperature; and
a therapeutic agent included in the waxy base.

18. The suppository of claim 17, wherein the therapeutic agent is an antibiotic.

19. The suppository of claim 17, wherein the therapeutic agent is a polyphosphate.

20. The suppository of claim 17, wherein the waxy base of the suppository is formed from glycerin.
